(19) United States
(12) Patent Application Publication
(10) Pub. No.: US 2025/0229103 A1
(43) Pub. Date: Jul. 17, 2025

(54) TREATMENT PLAN EVALUATION SYSTEM, TREATMENT PLAN EVALUATION METHOD, AND RADIOTHERAPY SYSTEM PROVIDED WITH TREATMENT PLAN EVALUATION SYSTEM

(71) Applicants: Hitachi High-Tech Corporation, Tokyo (JP); NATIONAL UNIVERSITY CORPORATION HOKKAIDO UNIVERSITY, Sapporoshi, Hokkaido (JP)

(72) Inventors: Takahiro YAMADA, Tokyo (JP); Yusuke FUJII, Tokyo (JP); Seishin TAKAO, Tokyo (JP); Koichi YASUDA, Tokyo (JP); Koichi MIYAZAKI, Tokyo (JP); Keiji KOBASHI, Tokyo (JP)

(21) Appl. No.: 18/850,607

(22) PCT Filed: Mar. 10, 2023
(86) PCT No.: PCT/JP2023/009271

§ 371 Date: Sep. 25, 2024

(30) Foreign Application Priority Data
Apr. 25, 2022 (JP) 2022-071855

(51) Int. Cl. A61N 5/10; G16H 20/40

(52) U.S. Cl. CPC A61N 5/103; G16H 20/40; A61N 2005/1041

(57) ABSTRACT

To provide a treatment plan evaluation system capable of supporting determination when a treatment plan is selected. A treatment plan evaluation system 1 that evaluates a treatment plan of a radiotherapy system RTS has a function of comparing a selected treatment plan with a record in which adoption of a past treatment plan has been determined, and provides a comparison result between the selected treatment plan and the record.

### TECHNICAL FIELD

The present invention relates to a treatment plan evaluation system, a treatment plan evaluation method, and a radiotherapy system provided with the treatment plan evaluation system.

### BACKGROUND ART

Online adaptive radiotherapy is a treatment method in which a treatment plan is recreated on the spot in accordance with the internal condition of a patient on the treatment day. PTL 1 discloses, as a workflow of online adaptive radiotherapy, automatically executing steps of acquiring a command representing a treatment plan, generating a patient model in stages by using the command, generating first and second treatment plans, and selecting the treatment plan.

### CITATION LIST

### Patent Literature

PTL 1: US 2020/0121951 A

### SUMMARY OF INVENTION

### Technical Problem

There is known a treatment method of irradiating a target volume such as cancer with a particle beam or radiation such as an X-ray. Examples of the particle beam include a proton beam and a carbon beam. In a radiation system used for irradiation with radiation, a dose distribution suitable for the shape of a target such as a tumor is formed. A patient is fixed on a patient bed called a couch and is irradiated with radiation to form a dose distribution suitable for the shape of a target inside the body of the patient.

The internal condition of the patient changes daily, such as a change in the shape of the target, a change in gas pockets in an intestinal tract, and the like. In order to improve the accuracy of irradiation with radiation, adaptive treatment for recreating a treatment plan in accordance with the internal condition of the patient on the treatment day has been spreading. On the treatment day, in a state where a patient is fixed to a couch, a treatment for replanning the treatment plan is called online adaptive treatment.

PTL 1 discloses an automatic workflow of the online adaptive treatment. In this workflow, steps of fixing a patient, imaging a target volume, creating a contour of the target volume, creating a treatment plan, selecting a treatment plan, and performing irradiation with radiation are sequentially performed. In the conventional treatment, the treatment is performed in three steps of fixing a patient, imaging a target volume, and performing irradiation with radiation. On the other hand, in the online adaptive treatment, since the number of steps to be performed is increased, there is a possibility that the treatment time is increased.

Further, on the treatment day, an operator is required to determine whether to use a treatment plan created first or a treatment plan created on the treatment day, for treatment. In this selection, an index of a dose distribution of the first treatment plan and an index of a dose distribution of the treatment plan created on the treatment day of are compared according to a plurality of criteria.

These criteria are set for each target or normal tissue. Even if there is a treatment plan in which some criteria do not achieve the goal, the treatment plan may be adopted by clinical judgment. However, in a case where some criteria do not achieve the goal, it becomes difficult to uniquely select either the first treatment plan or the treatment plan created on the treatment day, and it may take time to make that determination.

The present invention has been made in view of the above circumstances, and an object of the present invention is to provide a treatment plan evaluation system, a treatment plan evaluation method, and a radiotherapy system provided with the treatment plan evaluation system, which are capable of supporting determination when a treatment plan is selected.

### Solution to Problem

In order to solve the above problems, according to the present invention, there is provided a treatment plan evaluation system that evaluates a treatment plan of radiotherapy. The treatment plan evaluation system has a function of comparing a selected treatment plan with a record in which adoption of a past treatment plan has been determined, and provides a comparison result between the selected treatment plan and the record.

### Advantageous Effects of Invention

According to the present invention, it is possible to compare the selected treatment plan with a record in which the adoption of the past treatment plan has been determined.

### BRIEF DESCRIPTION OF DRAWINGS

FIG. 1 is an overall configuration diagram of a radiotherapy system.

FIG. 2 is a configuration diagram of a replanning system.

FIG. 3 is a flowchart illustrating processing of online adaptive treatment.

FIG. 4 is a flowchart illustrating details of processing of selecting a treatment plan in FIG. 3.

FIG. 5 is a screen example for providing information for supporting determination of selection of a treatment plan.

FIG. 6 is a screen example for providing a result of classifying treatment results.

FIG. 7 is another screen example for providing the result of classifying the treatment results.

FIG. 8 is a flowchart illustrating processing of online adaptive treatment according to a second embodiment.

FIG. 9 is a configuration diagram of a radiotherapy plan evaluation system according to a third embodiment.

### DESCRIPTION OF EMBODIMENTS

Hereinafter, embodiments of the present invention will be described with reference to the drawings. In the present embodiment, the selected treatment plan is compared with a record in which the adoption of the past treatment plan has been determined, and a comparison result between the selected treatment plan and the record is provided. In the present embodiment, a radiotherapy system RTS capable of performing adaptive treatment in a short time is provided. For this purpose, the radiotherapy system RTS includes a radiotherapy plan evaluation system 1. The radiotherapy plan evaluation system 1 classifies treatment plans based on a model for classifying the treatment plan, and provides a user 70 with the classification result of the treatment plan. According to the present embodiment, it is possible to evaluate each of a first treatment plan and a new treatment plan created on the treatment day. Since the user 70 can determine which of a plurality of treatment plans to appropriately select in a short time based on the evaluation result, it is possible to reduce the time required for the adaptive treatment, and to reduce the burden on a patient.

### Embodiment 1

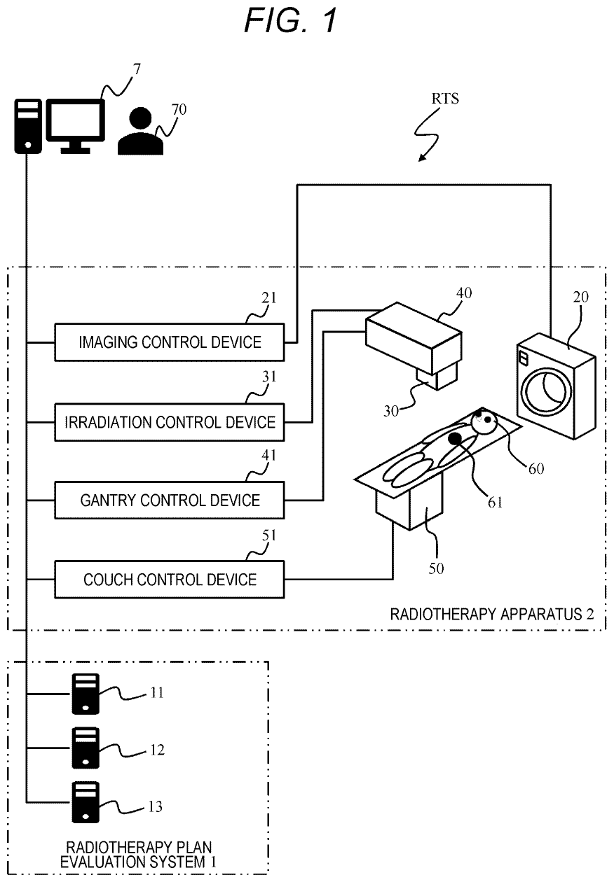

A first embodiment will be described with reference to FIGS. 1 to 7. The following description and drawings are merely examples, and are omitted and simplified as appropriate for clarity of description. The present invention is not limited to the configuration specified in the present disclosure. Unless otherwise specified, each component may be singular or plural.

Positions, sizes, shapes, ranges, and the like of components illustrated in the drawings may not represent actual positions, sizes, shapes, ranges, and the like. The present disclosure is not limited to the positions, sizes, shapes, ranges, and the like disclosed in the drawings. In a case where a plurality of components having the same or similar functions are provided, the same reference signs may be denoted with different subscripts for description. However, in a case where it is not necessary to distinguish the plurality of components from each other, the description may be made with the subscripts omitted.

FIG. 1 is an overall configuration diagram of a radiotherapy system RTS according to the present embodiment. The radiotherapy system RTS includes a radiotherapy plan evaluation system 1, a radiotherapy apparatus 2, and a workflow manager 7.

The radiotherapy plan evaluation system 1 is a system that classifies and evaluates a treatment plan used for radiotherapy by a model, and provides the evaluation result to an operator 70 as a “user” via the workflow manager 7. The radiotherapy plan evaluation system 1 includes, for example, a patient positioning system 11, a replanning system 12, and a patient verification (QA: quality assurance) system 13 as described later.

The radiotherapy apparatus 2 is an apparatus that irradiates a target volume (target) 61 of a patient 60 with radiation based on the selected treatment plan. As will be described later, the radiotherapy apparatus 2 includes, for example, an imaging device 20, an imaging control device 21, an irradiation nozzle 30, an irradiation control system 31, a rotating gantry 40, a gantry control device 41, a couch 50, and a couch control device 51.

A bed on which the patient 60 is placed is referred to as the couch 50. The couch 50 can move in directions of three orthogonal axes based on an instruction from the couch control device 51, and can further rotate about the respective axes. These movements and rotations can move the position of the target 61 to a desired position.

The imaging device 20 measures a three-dimensional image of the patient 60 and the target 61 fixed to the couch 50 based on an instruction from the imaging control device 21. The three-dimensional image is a CT image, a cone beam CT image, or an MRI image, and is referred to as a treatment day image below.

The irradiation nozzle 30 generates radiation used for treatment based on an instruction from the irradiation control system 31. dose Specifically, a desired distribution is formed for the target 61 by controlling the irradiation position and the irradiation dose of the radiation. A part of the irradiation nozzle 30 is installed in the rotating gantry 40 and can rotate together with the rotating gantry 40. The rotating gantry 40 is moved to a desired angle based on an instruction from the gantry control device 41. By changing the angle of the rotating gantry 40, irradiation with radiation can be performed from a desired angle.

A configuration of the radiotherapy plan evaluation system 1 will be described. The patient positioning system 11 calculates a position correction amount of the patient 60 with respect to the irradiation nozzle 30 based on the CT image at the time of treatment plan (an image at the time of treatment plan) and the treatment day image. The operator 70 confirms the calculation result and determines the position correction amount. Based on the determined position correction amount, the installation position of the couch 50 is set in the couch control device 51, and the position of the couch 50 is corrected.

The replanning system 12 generates a composite CT image used for replanning based on the image at the time of treatment plan and the image on the treatment day. Replanning is to re-create a treatment plan. The replanning system 12 further specifies areas of the target and a normal tissue on the composite CT image and creates contour data thereof. The replanning system 12 optimizes irradiation parameters of the radiation based on the composite CT image and the contour data to create a treatment plan.

A treatment plan created on the treatment day is referred to as the latest plan below. The original treatment plan created before the treatment day is referred to as the original plan. Since the latest plan is a treatment plan created on the treatment day, the latest plan can also be referred to as a daily plan.

The replanning system 12 provides the dose distribution of the latest plan to the operator 70 via a screen of the workflow manager 7. Based on the provided information, the operator 70 determines whether or not to use the latest plan for treatment on that day.

The patient QA system 13 verifies the latest plan and provides the verification result to the operator 70 via the screen of the workflow manager 7. The operator 70 confirms and approves the verification result of the latest plan.

The workflow manager 7 is bidirectionally communicatively connected to the imaging control device 21, the irradiation control system 31, the gantry control device 41, the couch control device 51, the patient positioning system 11, the replanning system 12, and the patient QA system 13, and monitors and manages the progress of a treatment workflow.

The workflow manager 7 is, for example, a computer terminal. The workflow manager 7 includes an input device for inputting various parameters and the like, a display device, a memory (storage medium), a database (storage medium), an arithmetic processing device (a control device that is an arithmetic element) that monitors and manages the progress of the workflow, and a communication device (none illustrated). The workflow manager 7 is configured as a desktop personal computer, a laptop personal computer, a tablet personal computer, a portable information terminal (including so-called a smartphone), a glasses wearable device, or the like.

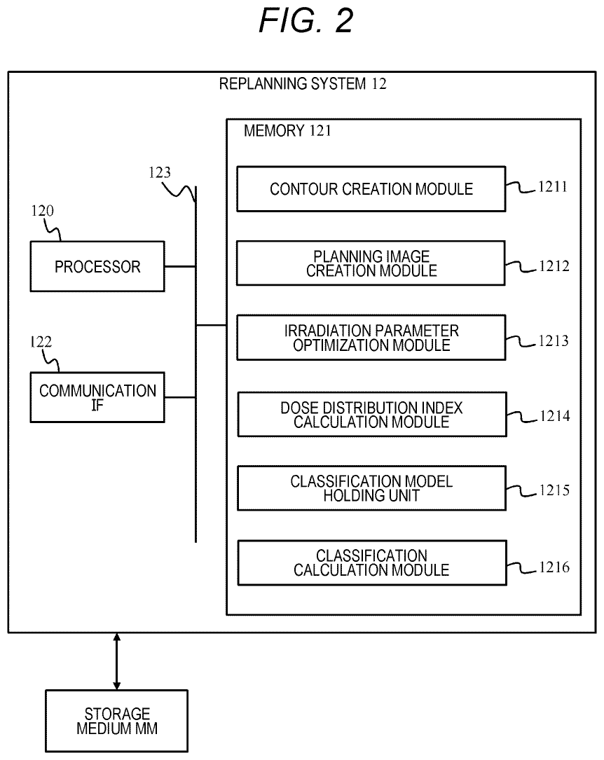

In FIG. 2 to be described later, an example in which the replanning system 12 is configured as a computer system will be described. The workflow manager 7, the patient positioning system 11, and the patient verification system 13 are also configured as computer systems as illustrated in FIG. 2. However, computer programs and data stored in the memory are different for each of the workflow manager 7, the patient positioning system 11, and the patient verification system 13.

FIG. 2 is a configuration diagram of the replanning system 12. The replanning system 12 is configured as a computer system, similarly to the workflow manager 7. The replanning system 12 includes, for example, a processor 120 as an arithmetic device, a memory 121 as a storage device, and a communication interface (in the drawing, a communication IF) 122 as a communication device.

The processor 120 is, for example, a central processing unit (CPU), a graphic processing unit (GPU), a field-programmable gate array (FPGA), or the like. The memory 122 here includes a main storage device and an auxiliary storage device. The memory 122 includes, for example, a magnetic storage medium such as a hard disk drive (HDD), a semiconductor storage medium such as a random access memory (RAM), a read only memory (ROM), and a solid state drive (SSD), and the like. A combination of an optical disk such as a digital versatile disk (DVD) and an optical disk drive is also used as a storage medium. In addition, a storage medium such as a magnetic tape medium may be used.

The memory 121 stores firmware, an operating system, and computer programs such as predetermined computer programs 1211 to 1216. When the operation of the replanning system 12 is started (for example, when the power is turned on), the processor 120 reads and executes a computer program such as firmware from the memory 121 and implements a function required as the replanning system 12. The processor 120 and the memory 122 are connected to the communication interface 123 via communication means 123 such as an internal bus.

The predetermined computer program stored in the memory 121 of the replanning system 12 includes, for example, a contour creation module 1211, a planning image creation module 1212, an irradiation parameter optimization module 1213, a dose distribution index calculation module 1214, a classification model holding unit 1215, and a classification calculation module 1216.

The contour creation module 1211 is a computer program that creates a contour of a target volume, a peripheral organ, and the like based on a CT image. The planning image creation module 1212 is a computer program that creates an image for creating a treatment plan. The irradiation parameter optimization module 1213 is a computer program that optimizes each parameter in the case of irradiating a target volume with radiation. The dose distribution index calculation module 1214 is a computer program that calculates an index of a dose distribution. The classification model holding unit 1215 is a computer program that holds a model for classifying appropriateness/inappropriateness of a treatment plan. The classification calculation module 1216 is a computer program that classifies the input treatment plan by using the model held by the classification model holding unit 1215.

A storage medium MM is, for example, a storage device capable of long-time storage, such as a flash memory or a hard disk drive. All or part of the computer program and data stored in the memory 121 can be transferred to and stored in the storage medium MM. The storage medium MM may be connected to a computer (not illustrated), and all or part of the computer program and data stored in the storage medium MM may be transferred to and stored in the computer (not illustrated).

Some functions of the replanning system 12 may be distributed and disposed in another computer, and the computer and the replanning system 12 may be connected via a communication network. That is, the replanning system 12 can be constructed by linking a plurality of computers. Note that, as will be described in an embodiment to be described later, the radiotherapy plan evaluation system 1 can also be provided in one computer.

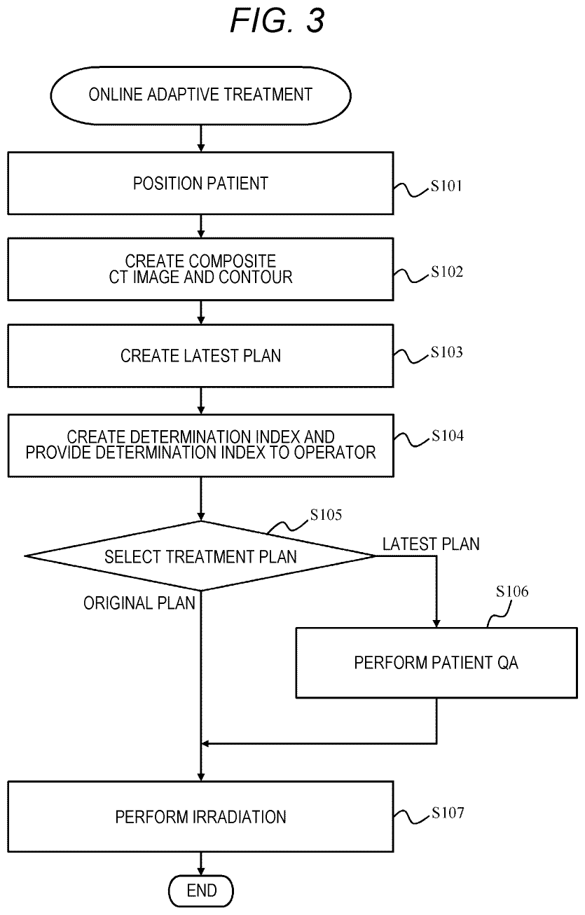

FIG. 3 is a flowchart of online adaptive treatment processing. First, the patient 60 is fixed to the couch 50, and an image of the treatment day is acquired by using the imaging device 20. The patient positioning system 11 corrects the position of the patient 60 based on a treatment plan image and an image (daily image) captured on the day (S101).

The patient positioning system 11 specifies areas of the target and a normal tissue based on the treatment day image and creates contour data thereof (S102). Furthermore, the patient positioning system 11 generates a composite CT image used for replanning based on the treatment plan image and the treatment day image (S102). For example, the treatment plan image is deformed from the treatment day image by using non-rigid registration DIR (Deformable Image Registration) to generate a composite CT image. At that time, the created contour data may be used.

In Step S103, the irradiation parameter optimization module 1213 obtains the irradiation parameter for realizing a target distribution by optimization calculation, based on the composite CT image and the contour data.

The dose distribution index calculation module 1214 calculates and displays the dose distribution in a case where irradiation with the radiation is performed according to the irradiation parameter obtained by the optimization calculation (S104).

In Step S105, the input of a plan selected by the operator 70 is received, and it is confirmed which treatment plan has been selected. The operator 70 selects a treatment plan to be used for treatment on that day, based on the dose distribution and dose distribution index of the original plan and the dose distribution and dose distribution index of the latest plan.

Here, the dose distribution index is an index calculated from a dose volume histogram (DVH) such as D95 indicating a dose including 95% of the volume of the target, V20 indicating a ratio of a volume irradiated with 20 Gy or more to the total volume, and average lung dose MLD. The dose distribution index may be a tumor control probability (TCP) or a normal tissue complication probability (NTCP). Details of Step S105 will be described later.

In a case where the latest plan is selected in Step S105, the process proceeds to Step S106. In Step S106, the latest plan is verified by the patient QA system 13, and the operator 70 confirms the verification result.

The operator 70 instructs the radiotherapy apparatus 2 according to the selected treatment plan to irradiate the target 61 with radiation (S107).

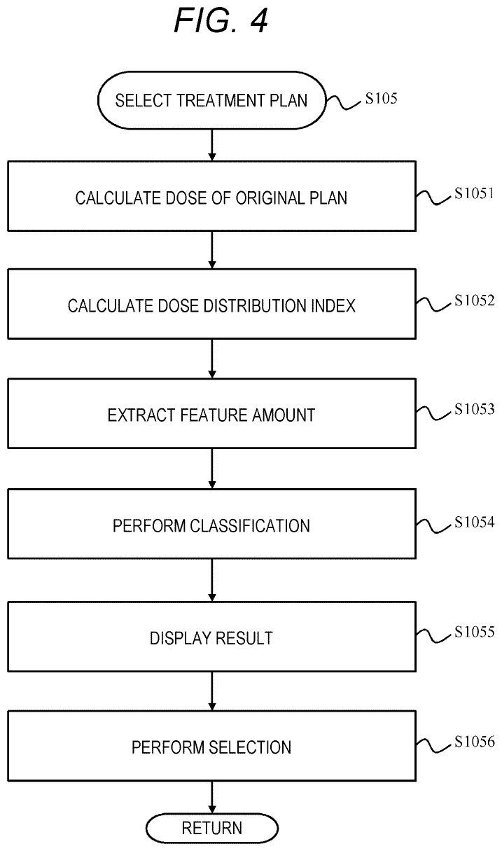

A plan selection method in Step S105 will be described with reference to FIG. 4.

In Step S1051, the dose distribution index calculation module 1214 calculates the dose distribution in a case where the original plan is irradiated based on the composite CT image.

In Step S1052, the dose distribution index calculation module 1214 calculates the dose distribution index of the original plan from the calculated dose distribution.

With the classification model read from the classification model holding unit 1215, the respective feature amounts are extracted from the dose distribution index of the original plan and the dose distribution index of the latest plan (S1053). The feature amount is a parameter used for calculation in the subsequent steps, and may be obtained by extracting a representative index from the dose distribution index, or may be a new index calculated by combining the dose distribution indices.

The classification calculation module 1216 classifies the original plan and the latest plan based on the feature amount extracted in Step S1053, and calculates which of a plurality of groups determined in advance the original plan and the latest plan belong to (S1054). The plurality of groups is determined based on a record of the past treatment.

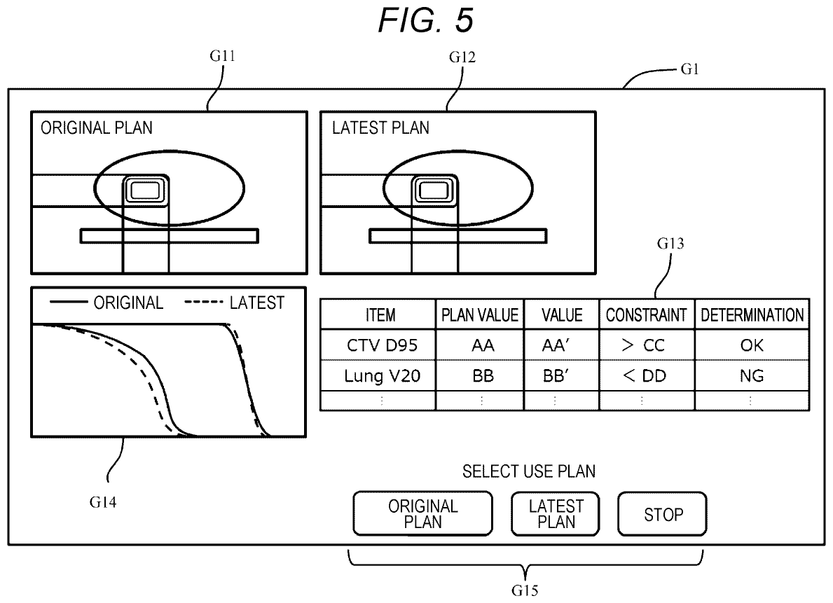

Reference is made to FIG. 5 here. In Step S1055, as illustrated in FIG. 5, a screen G1 indicating the calculation result of the dose distribution is displayed on the workflow manager 7. FIG. G11 illustrates the dose distribution of the original plan. FIG. G12 illustrates the dose distribution of the latest plan. In Table G13, the dose distribution index of the original plan and the dose distribution index of the latest plan are displayed. In FIG. G14, DVH of the original plan and DVH of the latest plan are displayed.

The operator 70 can change the positions and contents displayed in FIG. G11 and FIG. G12, respectively. The position and the content displayed in FIG. G11 and the position and the content displayed in FIG. G12 can be interlocked. That is, in a case where the position or the content displayed in any one of the figures is changed, the position and the content displayed in the other figure may be similarly changed. The position and the content displayed in FIG. G11 and the position and the content displayed in FIG. G12 may be independent of each other.

The dose distribution index is displayed in Table G13 together with whether or not a standard set in advance is achieved. Furthermore, on the screen G1, a button G15 for the operator 70 to select a treatment plan to be used for treatment is displayed.

Returning to FIG. 4. In Step S1056, the operator 70 confirms the classification result provided by the replanning system 12, and selects which of the original plan and the latest plan is to be used for treatment.

An example of a method of creating the classification model will be described. The classification model is created based on the dose distribution index when the necessity of replanning has been determined in the past. For example, in a conventional treatment that is not an online adaptive treatment, there is a case where a dose distribution in a case where an original treatment plan is applied to a captured CT image is calculated during a treatment period, and necessity of replanning is determined based on a dose distribution index calculated from the dose distribution. The classification model is constructed by using, as teacher data, a combination of a dose distribution index at the time of determining the necessity of replanning and a result of whether or not the replanning has been actually performed.

Since there are several tens of dose distribution indices used for determination of necessity of replanning, the number of dose distribution indices for creating the classification model is reduced by selecting variables as necessary. By performing main component analysis based on the selected dose distribution index, a new feature amount can be generated. A method other than the main component analysis can also be used.

These procedures of variable selection and feature amount creation when the classification model is created are stored in the classification model holding unit 1215. At the time of plan selection in adaptive treatment, similar calculation is performed in Step S1053. The classification model is created by using, as teacher data, a combination of the feature amount extracted in this manner and a result of whether or not the replanning has been actually performed.

The classification model may be created based on the dose distribution index when either the latest plan or the original plan is selected during the online adaptive treatment.

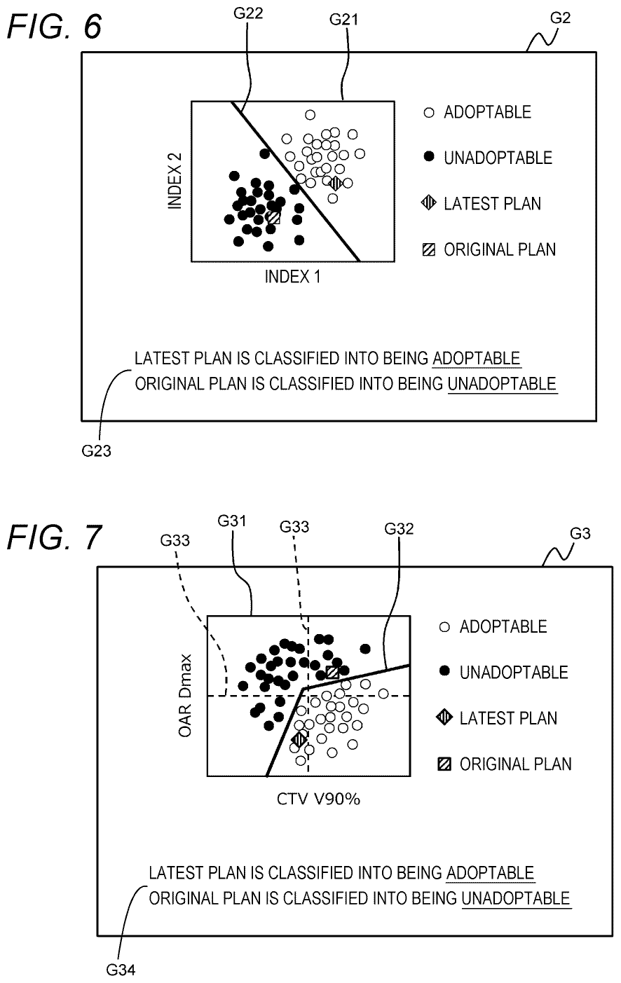

FIG. 6 illustrates an example of a classification result screen G2. On the classification result screen G2, a distribution G21 of the representative feature amounts and a classification boundary G22 are displayed. The feature amounts to be displayed in the feature amount distribution G21 that is an example of “information indicating the reason for adoptability” can be selected by the operator 70.

In the feature amount distribution G21, data used for creating the classification model is displayed in a distinguished manner so that whether the data is data in a case where replanning has been performed or data in a case where replanning is not performed can be understood. The data that has not been replanned is described as “adoptable”, for example. The replanned data is described as “unadoptable”, for example. The latest plan and the original plan are also displayed separately in the distribution G21. Furthermore, the classification results of the latest plan and the original plan are displayed as a sentence G23 as an example of “information indicating adoptability”.

Another classification result screen G3 is illustrated with reference to FIG. 7. On the classification result screen G3, a distribution diagram G31 of representative dose distribution indices and a classification boundary G22 are displayed. The dose distribution index displayed in the distribution G31 can be freely selected by the operator 70. In the distribution G31, a reference G33 set for each guideline or facility is also displayed.

In the case of FIG. 7, CTV V90% is better than the reference, and OAR Dmax is better than the reference. The displayed points are distinguished and displayed so that it can be understood whether the data used for creating the classification model is data in a case where replanning has been performed or data in a case where replanning is not performed. The data that has not been replanned is described as “adoptable”, for example. The replanned data may be described as “unadoptable”, for example. The latest plan and the original plan are also displayed separately in the distribution G31. Further, the classification results of the latest plan and the original plan are displayed as a sentence G34.

The feature amount distribution method, whether or not to perform replanning, the sentence of the classification result, and the like are not limited to the examples illustrated in FIGS. 6 and 7.

According to the present embodiment configured as described above, information for evaluating the selected treatment plan can be provided to the operator 70 by using the model for classifying the treatment plan. As a result, it is possible to support determination when the operator selects a treatment plan, and it is possible to improve usability for the operator. Furthermore, since the time required for the operator to select the treatment plan can be shortened, it is possible to also shorten the treatment time of the patient on the treatment day, and to reduce the burden on the patient.

In the radiotherapy system RTS using the treatment plan evaluation system 1 in the present embodiment, the situation of the target 61 of radiotherapy and the normal tissue existing around the target changes every day. For example, in the abdomen and the like, the positions and the sizes of the target 61 and the normal tissue vary every day depending on the situation such as the position or contents of the digestive tract.

For example, in treatment of uterine body cancer, bladder, rectum, small intestine, and large intestine are normal tissues existing around the target. In treatment of pancreatic cancer, a spinal cord, a stomach, a duodenum, a liver, a kidney, and the like are normal tissues existing around the target. In a case where the normal tissue is close to the periphery of the target 61, even in the online adaptive treatment, satisfying both the reference of the dose distribution index for the target and the reference of the dose distribution index for the normal tissue is not possible in some cases.

For example, in a case where the position of the target 61 is closer to the position of the OAR than at the time of treatment plan, as illustrated in the distribution diagram G31 of FIG. 7, in some cases, the criteria of the CTV are achieved in the original plan, but the criteria of the OAR cannot be achieved.

On the other hand, even with the latest plan replanned based on the image on the treatment day, the OAR criteria may be achieved but the CTV criteria may not be achieved due to the proximity of the OAR. In a case where such a situation occurs in both the latest plan and the original plan, it becomes difficult to uniquely select any treatment plan, and it takes time to make a determination.

However, in the present embodiment, since the latest plan and the original plan can be evaluated based on the record of determination for the past treatment, the operator can easily select a plan to be used on the treatment day. Then, in the present embodiment, since the determination of the operator can be supported, it is possible to shorten the treatment time.

### Embodiment 2

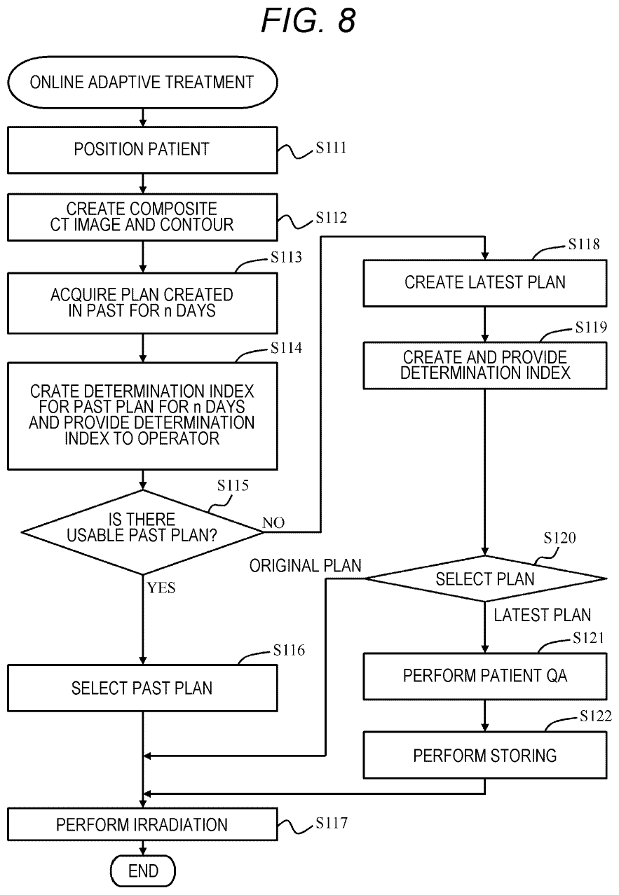

A second embodiment will be described with reference to FIG. 8. In the present embodiment, a treatment plan used in radiotherapy for a certain patient is stored (S122). It is determined whether there is a reusable treatment plan among treatment plans adopted in the past (S115). In a case where there is no reusable treatment plan, a latest plan is created (S118).

As described with reference to FIG. 3, first, the patient 60 is fixed to the couch 50, and an image of the treatment day is acquired by using the imaging device 20. The patient positioning system 11 corrects the position of the patient 60 based on a treatment plan image and an image (daily image) captured on the day (S111). The patient positioning system 11 specifies areas of the target and a normal tissue based on the treatment day image and creates contour data thereof (S112). The patient positioning system 11 generates a composite CT image used for replanning based on the treatment plan image and the treatment day image (S112).

The replanning system 12 acquires plans (treatment plans) created in the past for the treatment of the patient for n days (S113). For the first treatment, n is 0. In the case of the second treatment which is started in a state where a new treatment plan is created in the first treatment, n is 1.

The replanning system 12 generates a determination index as described with reference to FIGS. 6 and 7 for the acquired plans for the past n days, and provides the determination index to the operator (S114). At that time, the replanning system 12 compares the determination index of the original plan with the determination index created after the original plan, and provides the result to the operator. That is, the replanning system 12 selects n plans one by one up while incrementing to n, compares the evaluation result obtained by classifying the selected plan with the classification model with the evaluation result obtained by classifying the original plan with the classification model, and provides the result to the operator (S114).

The operator determines whether there is a plan that can be used for today's treatment among plans created in the past based on determination support information (information illustrated in FIGS. 5, 6, and 7 as an example thereof) provided from the replanning system 12. The operator inputs the determination result from the workflow manager 7 to the replanning system 12.

When the operator determines that any one of the plans created in the past can be used, the selection result by the operator is input to the replanning system 12 (S115: YES, S116). Then, the operator operates the radiotherapy apparatus 2 according to the selected past plan (S117).

On the other hand, in a case where the operator determines that no plan that can be used for today's treatment is found among the plans created in the past, the determination result is input to the replanning system 12 (S115: NO).

As described in Steps S103 to S106 in FIG. 3, the replanning system 12 creates a latest plan (S118), creates a determination index and provides the determination index to the operator (S119), and receives the selection result of the plan by the operator (S120). In a case where the original plan is selected, radiotherapy is performed according to the original plan (S117). In a case where the latest plan is selected, the patient QA is performed for the latest plan (S121), and the latest plan for which the patient QA is performed is stored in the replanning system 12 (S122). Then, radiotherapy is performed according to the latest plan (S117).

The present embodiment configured as described above also has the similar operational effects to those of the first embodiment. Furthermore, in the present embodiment, before the latest plan on the treatment day is created, it is determined whether the past plan created for the patient cannot be reused, and, in a case where the past plan can be used, the past plan is used. Therefore, the time until the radiotherapy is started may be shortened as compared with the first embodiment, and thus it is possible to reduce the burden on the patient. In the radiotherapy system RTS, radiotherapy is performed a plurality of times such as several tens of times or several tens of times on the same target volume of the same patient, and thus, there is a probability that the plan created in the past can be reused. In the present embodiment, the time required for selecting a plan is shortened by using the special property of radiotherapy in which a target of which a relationship with surrounding normal tissues and the like changes every day is irradiated with radiation a plurality of times over time.

### Embodiment 3

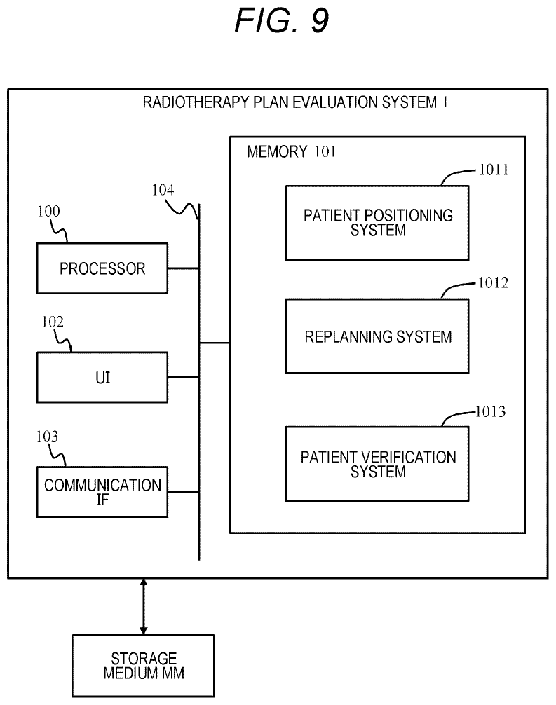

A third embodiment will be described with reference to FIG. 9. In the present embodiment, a case where the radiotherapy plan evaluation system 1 is configured by one computer system will be described.

The computer system includes, for example, a processor 100, a memory 101, a user interface unit 102, and a communication interface unit 103. The memory 101 stores, for example, a computer program 1011 for realizing the function of the patient positioning system 11, a computer program 1012 for realizing the function of the replanning system 12, and a computer program 1013 for realizing the patient verification system 13.

The present embodiment configured as described above also has the similar operational effects to those of the first embodiment. In the present embodiment, since the radiotherapy plan evaluation system 1 is configured as one computer system, it is possible to simplify the configuration of the radiotherapy system RTS.

Note that the present invention is not limited to the above embodiments, and includes various modifications. The above embodiments have been described in detail in order to describe the present invention in an easy-to-understand manner, and are not necessarily limited to those having all the described configurations.

Some or all of the above configurations, functions, processing units, processing means, and the like may be realized by hardware, for example, by designing with an integrated circuit. In addition, each of the above configurations, functions, and the like may be realized by software by a processor interpreting and executing a program for realizing each function. Information such as a program, a table, and a file for realizing each function can be stored in a recording device such as a memory, a hard disk drive, or an SSD, or a recording medium such as an IC card, an SD card, or a DVD.

In addition, the control lines and the information lines indicate what is considered to be necessary for the description, and do not necessarily indicate all the control lines and the information lines on the product. It may be considered that almost all the configurations are actually connected to each other.

The present specification discloses an invention of a computer program for realizing the treatment plan evaluation system as follows.

“A computer program that causes a computer to function as a treatment plan evaluation system that evaluates a treatment plan of radiotherapy, the computer program causing the computer to hold a model for classifying a treatment plan, classify a selected treatment plan by the model, and provide an evaluation result obtained by classifying the selected treatment plan by the model to a terminal used by a user, or a storage medium that stores the computer program.”

### REFERENCE SIGNS LIST

1 radiotherapy plan evaluation system
2 radiotherapy apparatus
7 workflow manager
11 patient positioning system
12 replanning system
13 patient verification system
20 imaging control device
21 imaging control device
30 irradiation nozzle
31 irradiation control system
40 rotating gantry
41 gantry control device
50 couch
51 couch control device

1. A treatment plan evaluation system that evaluates a treatment plan of radiotherapy, wherein
a function of comparing a selected treatment plan with a record in which adoption of a past treatment plan has been determined is provided, and
a comparison result between the selected treatment plan and the record is provided.

2. The treatment plan evaluation system according to claim 1, wherein a classification model based on the record on the adoption of the past treatment plan is held,
a function of classifying the selected treatment plan by the model is provided, and
an evaluation result obtained by classifying the selected treatment plan by the model is provided.

3. The treatment plan evaluation system according to claim 2, wherein the selected treatment plan includes a first treatment plan created earlier and a second treatment plan created later than the first treatment plan.

4. The treatment plan evaluation system according to claim 2, wherein the selected treatment plan includes a first treatment plan created first and n (n is a natural number of 2 or more) second treatment plans each created at a time after the first treatment plan, and
the n second treatment plans are selected one by one while incrementing to n, and an evaluation result obtained by classifying the selected second treatment plan by the model and an evaluation result obtained by classifying the first treatment plan by the model are provided.

5. The treatment plan evaluation system according to claim 3, wherein an evaluation result obtained by classifying the first treatment plan by the model and an evaluation result obtained by classifying the second treatment plan by the model are provided in comparison with each other.

6. The treatment plan evaluation system according to claim 3, wherein the evaluation result obtained by being classified by the model includes information indicating adoptability and information indicating a reason for the adoptability.

7. The treatment plan evaluation system according to claim 2, wherein the treatment plan is a plan in which a target volume and a normal tissue are close to each other and irradiation with radiation is performed according to a plurality of predetermined dose distribution indices.

8. The treatment plan evaluation system according to claim 2, wherein the radiotherapy is online adaptive radiotherapy.

9. A radiotherapy system that performs irradiation with radiation, wherein
a target volume is irradiated with the radiation according to a treatment plan evaluated and selected by the treatment plan evaluation system according to claim 2.

10. A treatment plan evaluation method for evaluating a treatment plan of radiotherapy by a computer, the method comprising:
by the computer,
comparing a selected treatment plan with a record in which adoption of a past treatment plan is determined; and
providing a comparison result between the selected treatment plan and the record to a terminal used by a user.
